(19) United States
(12) Patent Application Publication
(10) Pub. No.: US 2013/0302365 A1
(43) Pub. Date: Nov. 14, 2013

(54) EXTRACTION OF OIL-SOLUBLE PIGMENT FROM MICRO-ORGANISMS

(71) Applicants: Andreas Weber, Weesp (NL); Nieves Gonzalez Ramon, Delft (NL)

(72) Inventors: Andreas Weber, Weesp (NL); Nieves Gonzalez Ramon, Delft (NL)

(73) Assignee: FEYECON B.V.

(21) Appl. No.: 13/883,968

(22) PCT Filed: Nov. 8, 2011
(86) PCT No.: PCT/NL2011/050761

§ 371 Date: Jul. 17, 2013

(30) Foreign Application Priority Data
Nov. 8, 2010 (EP) 10190360.7

(51) Int. Cl. A23L 1/275; C07C 7/10; C07D 301/32; C07C 45/80; C07C 29/86

(52) U.S. Cl. CPC A23L 1/2755; C07C 45/80; C07C 29/86; C07D 301/32; C07C 7/10
USPC 424/195.17; 426/655; 426/540; 426/429

(57) ABSTRACT

A microalgae extract is disclosed consisting of: (A) 40-95 wt. % of a polar phase comprising: (i) 15-75 wt. % water; (ii) 20-80 wt. % of alcohols, acetone and/or combinations thereof; (iii) 0-10 wt. % of sodium chloride; (iv) up to 40 wt. % of other water soluble components; (B) 5-60 wt. % of an apolar phase comprising: (i) 70-99.5 wt. % of triglycerides, diglycerides, monoglycerides, phosphatides, free fatty acids and/or combinations thereof; (ii) 0.05-25 wt. % of oil-soluble pigment; (iii) up to 30 wt. % of other oil soluble components; and (iv) optionally other components in a concentration that does not exceed 9% by weight of the water contained in the extract, wherein the extract contains less than 0.5% by weight of the apolar phase of apolar organic solvent selected from C₅-C₁₀ alkanes. Processes for extracting an oil-soluble pigment from wet biomass of micro-organisms are also disclosed.

### TECHNICAL FIELD OF THE INVENTION

The present invention relates to a micro-algae extract containing carotenoids and/or other oil-soluble pigments, such as chlorophylls. The invention also provides processes for extracting such oil-soluble pigment from micro-organisms, e.g. from wet biomass of micro-algae.

### BACKGROUND OF THE INVENTION

Carotenoids are natural pigments that are responsible for many of the yellow, orange, and red colors seen in living organisms. Carotenoids are widely distributed in nature and perform a number of important biological functions, including protecting organisms from photo-oxidative damage. Carotenoids are generally split into two classes, xanthophylls (which contain oxygen) and carotenes (which are purely hydrocarbons, and contain no oxygen). Extracted and/or synthetic carotenoids can be used as nutraceuticals, dietary supplements, or pharmaceuticals. For example, astaxanthin is known to have a potent antioxidative effect.

Microphytes or microalgae are microscopic algae, typically found in freshwater and marine systems. The biodiversity of microalgae is enormous and they represent an almost untapped resource. Most of these microalgae species produce significant amounts of various kinds of, commercially interesting, carotenoids. Numerous methods of optimizing the production of such carotenoid-rich biomasses and improving carotenoid-recovery there from have been attempted over the years. However the main source for commercial carotenoids is still synthetic. This is mainly due to difficulties encountered in the process of extracting carotenoids and other oil-soluble pigments from biomass.

Ideally, extraction of oil-soluble pigment from micro-organisms should be fast, efficient and gentle.

The extraction solvents used should be inexpensive, volatile (for easy removal), free from toxic or reactive impurities (to avoid reaction with e.g. lipids), able to form a two-phase system with water (to remove non-lipids), and be poor extractors of unwanted components. Currently, liquid organic solvents are frequently used to extract carotenoids and other pigments from natural substrates. Most of the methods employ apolar solvents such as hexane, acetone, chloroform or diethyl ether. However these solvents are losing favor as extractants because their handling poses a safety hazard as they are highly explosive and/or toxic. In any event, after such extraction with organic solvents, distillation is required to remove the organic solvent(s) from the extracted carotenoid fractions as only trace levels of these solvents are allowed in food ingredients. The high temperature required for distilling the final solvent remnants can degrade the sensitive extract. Hence there is need for a more attractive alternative.

Solvent extraction of microalgae is known in the art. WO 2010/104922 describes a method of fractionating biomass, including the steps of: permeability conditioning biomass suspended in a pH adjusted solution of at least one water-based polar solvent to form a conditioned biomass, intimately contacting the conditioned biomass with at least one non-polar solvent, partitioning to obtain a non-polar solvent solution and a polar biomass solution, and recovering cell products from the non-polar solvent solution and polar biomass solution. Examples 2 and 8 of WO 2010/104922 describe a process in which Nannochloropsis sp. is resuspended in water, pH was adjusted to 2.0 and the mixture was heated to 60° C. for 15 minutes before extraction with hexane and wherein the lipid containing hexane extract was distilled to remove hexane.

WO 2009/144830 describes a method of treating a biomass source, comprising:
contacting the biomass source with a co-solvent composition, wherein the co-solvent composition comprises at least one ionic liquid and at least one polar covalent molecule (PCM); and
mixing the co-solvent composition and the biomass source, wherein said contacting and mixing results in formation of a multiple-phase composition, wherein a first phase comprises a first biomass component that is immiscible with the co-solvent composition.
   Example 2 of O 2009/144830 describes direct transesterification of the oil contained in dried Nannochloropsis cells in the presence of excess methanol and acid catalyst, followed by extraction with hexane

Several techniques for extraction of pigments from microalgae based on other solvents have been described in the prior art.

EP-A 612 725 describes extraction of β-carotene from halophilic algae of the genus Dunaliella including D. salina, D. parva, D. tertiolecta, D. primolecta and D. peircei. An aqueous suspension of the biomass is emulsified with edible oil (soya bean oil, peanut oil, or sunflower seed oil) at elevated temperatures and the mixture is subjected to membrane ultra-filtration.

Extraction of β-carotene from natural sources using as solvent one of acetone, methyl-ethyl ketone, methanol, ethanol, propan-2-ol, hexane, dichloromethane and supercritical carbon dioxide is described in U.S. Pat. No. 5,714,658, as is the use of a mixture of an acetic acid ester, such as ethyl acetate, and an edible oil.

WO 2001/46133 discloses the extraction of astaxanthin from various biomasses by treating a carotenoid source at temperatures of more than 50° C. with a solvent mixture comprising water, a hydrophobic carotenoid solvent, such as a vegetable oil, and a water-soluble co-solvent such as ethanol.

These and other existing methods for isolating pigments from microalgae suffer from a number of drawbacks, such as their relative complexity, especially the number of isolation steps to be employed and/or the application of heat and/or pressure. The application of heat, in addition to being impractical, has also been found to result in (partial) degradation of the desired product.

Accordingly, efforts to extract carotenoids from natural sources have not yet resulted in sufficiently cost-effective products, i.e. relative to their synthetic analogs. It is the object of the present invention to provide an improved extraction process, which may render commercial use of carotenoids from natural sources an economically viable option and that does not require the use of apolar organic solvents such as hexane.

### SUMMARY OF THE INVENTION

The present inventors have developed an alternative approach for isolating oil-soluble pigment, such as carotenoids and/or chlorophyll, from micro-organisms that avoids at least some of the aforementioned drawbacks.

The process according to the present invention starts from wet biomass, does not require the use of apolar organic solvents and produces an extract that can easily be downstream processed to produce a pigment-containing oil in high yield, or that may be used as such, e.g. in the production of a foodstuffs or animal feed.

The extract obtained by the present process comprises a polar phase and an apolar phase that together contain the bulk of the oil-soluble pigment that was originally contained in the wet biomass. Thus, one aspect of the present invention relates to a microalgae extract consisting of:
40-95 wt. % of a polar phase comprising:
   a. 15-75 wt. % water;
   b. 20-80 wt. % of an organic water miscible solvent selected from alcohols, acetone and combinations thereof;
   c. 0-10 wt. % of sodium chloride;
   d. up to 40 wt. % of other water soluble components;
5-60 wt. % of an apolar phase comprising:
   a. 70-99.5 wt. % of oil selected from triglycerides, diglycerides, monoglycerides, phosphatides, free fatty acids and combinations thereof;
   b. 0.05-25 wt. % of oil-soluble pigment;
   c. up to 30 wt. % of other oil soluble components; and
optionally other components, said optional other components being present in a concentration that does not exceed 9% by weight of the water contained in the extract
   wherein the extract contains less than 0.5% by weight of the apolar phase of apolar organic solvent selected from C₅-C₁₀ alkanes.

Another aspect of the invention relates to a process of extracting oil-soluble pigment from wet biomass of micro-organisms, especially microalgae, comprising:
combining the wet biomass with a water miscible organic solvent selected from alcohols, acetone and combinations thereof, said biomass containing 10-95 wt. % of water and at least 0.01% by weight of dry matter of oil-soluble pigment;
isolating a liquid polar phase from the combination of biomass and solvent, said polar phase containing 20-80 wt. % of the water miscible organic solvent, 15-75 wt. % of water and at least 0.01 wt. % of pigment originating from the biomass;
adding oil to the liquid polar phase in an amount of 5-150% by weight of the liquid phase to extract oil-soluble pigment from said liquid polar phase into the oil, said oil being selected from triglycerides, diglycerides, monoglycerides, phosphatides, free fatty acids and combinations thereof; and
separating the oil phase containing oil-soluble pigment from the extracted polar phase, said oil phase containing at least 0.05 wt. % of dissolved pigment originating from the biomass.

These and other aspects of the present invention will be described and illustrated in more detail hereafter.

### DETAILED DESCRIPTION OF THE INVENTION

Accordingly, a first aspect of the invention relates to a microalgae extract consisting of:
40-95 wt. % of a polar phase comprising:
   a. 15-75 wt. % water;
   b. 20-80 wt. % of an organic water miscible solvent selected from alcohols, acetone and combinations thereof;
   c. 0-10 wt. % of sodium chloride;
   d. up to 40 wt. % of other water soluble components;
5-60 wt. % of an apolar phase comprising:
   a. 70-99.5 wt. % of oil selected from triglycerides, diglycerides, monoglycerides, phosphatides, free fatty acids and combinations thereof;
   b. 0.05-25 wt. % of oil-soluble pigment;
   c. up to 30 wt. % of other oil soluble components; and
optionally other components, said optional other components being present in a concentration that does not exceed 9% by weight of the water contained in the extract
   wherein the extract contains less than 0.5% by weight of the apolar phase of apolar organic solvent selected from C₅-C₁₀ alkanes.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

As used herein the terms “apolar” and “ non-polar” are deemed to be synonymous and may thus be used interchangeably.

The term “microalgae” generally refers to unicellular algae species that are native to aquatic or marine habitats and that exist individually or in chains or groups. Microalgae generally have an average diameter of about 20 μm or less, preferably 15 μm or less, more preferably 10 μm or less, for example 5 μm or less.

The term “organic water-miscible solvent” encompasses organic solvents capable of forming a homogeneous blend with water within a wide range of mixing ratios.

The benefits of the present invention may be realized using all kinds of microalgae. According to a preferred embodiment, the microalgae employed in accordance with the present invention are not silicate or calcium secreting species that belong to the diatoms or the genus Coccolithophores. Examples of genus/classes of microalgae that can suitably be employed include Chysophyceae, Xantophyceae, Eustigmatophyceae, Baccilariophyceae, Dinophyceae, Rodophyceae, Phaeophyceae, Chlorophyceae, Prasinophyceae, Cryptophyceae, Botrycoccus, Isochrysis, Tetraselmis, Neochloris, Scenedesmus, Chlorobotrys, Eustigmatos, Pseudostaurastrum, Vischeria, Monodopsis, Ellipsoidion, Pseudocharaciopsis and combinations thereof.

The microalgae employed in accordance with the present invention preferably are green microalgae. Even more preferably, the microalgae employed, belongs to a genus or class selected from Eustigmatophyceae, Chlorophyceae and combinations thereof. Most preferably, the microalgae employed in accordance with the present invention are selected from Nannochloropsis gaditana, Isochrysis galbana, Chlorella fusca, Haematococcus pluvialis and combinations thereof.

The present microalgae extract can be obtained by subjecting a wet biomass containing said microalgae to the extraction process described herein below. The extract so obtained is typically a “biphasic extract”, which refers to an extract comprising at least a polar phase and an apolar phase. Thus, the term “biphasic extract” also encompasses emulsions comprising three or more phases, e.g. a water-in-oil-in-water emulsion or an emulsion containing another phase that is immiscible with the aqueous phase or the oil phase. According to a preferred embodiment, the microalgae extract according to the present invention is a non-emulsified biphasic extract, i.e. an extract comprising a layer of polar phase and a layer of apolar phase sitting on top said layer of polar phase.

According to a particularly preferred embodiment, the microalgae extract comprises 50-90 wt. % of the polar phase and 10-50 wt. % of an apolar phase. Most preferably, the microalgae extract comprises 60-80 wt. % of a polar phase and 20-40 wt. % of a non-polar phase. These percentages are relative to the total weight of the abstract. Typically, the polar phase and the apolar phase together represent at least 92 wt. %, preferably at least 95 wt. % and most preferably at least 98 wt. % of the extract.

Typically, the extract contains 20-70 wt. %, more preferably 25-65 wt. %, most preferably 30-60 wt. % of organic water-miscible solvents. It is furthermore preferred that the weight ratio of water to water-miscible solvent in the polar phase exceeds 1:2, more preferably it exceeds 3:4, most preferably it exceeds 1:1.

The apolar phase of the microalgae extract typically comprises 72-99.5 wt. % of oil, preferably 75-99 wt. %, more preferably 80-98 wt. %, most preferably 85-95 wt. % of oil or of an oil composition, relative to the total weight of the entire apolar phase.

According to another preferred embodiment, the oil contained in the apolar phase comprises at least 50 wt. %, more preferably at least 75 wt. % and most preferably at least 90 wt. % of triglycerides.

In one particularly preferred embodiment of the invention, an extract is provided as defined herein before, wherein the oil contained in the apolar phase comprises at least 50 wt. % of a vegetable oil or medium chain triglyceride oil, more preferably at least 50 wt. % of a vegetable oil selected from the group consisting of corn oil, rapeseed oil, soybean oil, palm oil, sunflower oil and coconut oil, safflower oil, peanut oil, coconut oil, sesame oil, soybean oil, olive oil, cottonseed oil, rice oil, linseed oil or mixtures thereof. The aforementioned vegetable oils or mixture of oils may suitably be employed in hydrogenated or interesterified form. The apolar phase of the present extract may suitably contain oil originating from microalgae biomass. Typically, the oil contained in the apolar phase comprises 1-25 wt. % of an oil of microalgae origin.

Furthermore, it is preferred that marine oil represents less than 50 wt. % of the oil contained in the apolar phase, preferably less than 35 wt. %, most preferably less than 25 wt. %.

The oil-soluble pigment contained in the present extract preferably is a pigment selected from the group consisting of carotenoids, chlorophyls and combinations thereof. Besides the latter oil-soluble pigments, other types of oil-soluble pigments may be contained in the present extract. More preferably, the oil-soluble pigment contained in the present extract is a carotenoid.

Xanthophylls represent a large proportion of the carotenoid pigments produced in algae. Examples of xanthophylls that are advantageously contained in the present extract include astaxanthine, zanthin, lutein, cryptoxanthin, Violaxanthin, diatoxanthin, diadinoxanthin fucoxanthin, neofucoxanthin A, and neofucoxanthin B dinoxanthin neodiadinoxanthin neodinoxanthin. Carotenes, such as β-carotene, represent another class of carotenoids that are advantageously contained in the present extract.

The apolar phase of the present extract preferably contains at least 0.3 wt. % of oil-soluble pigments, more preferably at least 0.5 wt. %, still more preferably at least 1.0 wt. %, most preferably at least 3.0 wt. %, based on the total weight of the apolar phase. Typically, the amount of oil-soluble pigments contained in the extract does not exceed 25% by weight of the apolar phase, preferably it does not exceed 20.% by weight of the apolar phase.

The apolar phase preferably contains at least 0.05 wt. % of carotenoids, more preferably at least 0.1 wt. %, still more preferably at least 0.3 wt. %, still more preferably at least 1.0 wt. %, most preferably at least 2.0 wt. % based on the total weight of the apolar phase. Typically, the amount of carotenoids contained in the extract does not exceed 25% by weight of the apolar phase, preferably it does not exceed 20% by weight of the apolar phase.

The microalgae used to produce the present extract preferably are green microalgae. Accordingly, in a preferred embodiment, the apolar phase contains at least 0.02 wt. %, more preferably 0.05 wt. % of chlorophyll by weight of the oil phase. Usually, the chlorophyll concentration of the extract does not exceed 1% by weight of the apolar phase.

The apolar phase can typically contain up to 20 wt. % of other oil soluble components, i.e. components other than oil or oil-soluble pigment. The apolar phase typically contains at least 0.01 wt. %, more preferably 0.1 wt. % of such other oil soluble components relative to the weight of the entire apolar phase. Usually, the amount of said other oil soluble components contained in the apolar phase of the extract does not exceed 10 wt. %, preferably it does not exceed 5 wt. %.

The polar phase of the present extract comprises water and an organic water-miscible solvent as the main components. Typically, water is present in an amount of 15-75 wt. % based on the total weight of the polar phase, preferably in amounts of 25-70 wt. %, more preferably 30-65 wt. % and most preferably 35-60 wt. %. It is furthermore preferred that the amount of water in the extract does not exceed 30 wt. %, on the basis of the weight of the entire extract, preferably it does not exceed 25 wt. %, more preferably it does not exceed 20 wt. %. Furthermore, the polar phase typically contains 20-80 wt. %, more preferably 25-70 wt. %, most preferably 30-60 wt. % of the water-miscible organic solvent, by weight of the polar phase.

Alkyl alcohols qualify as an organic water-miscible solvent, so long as their melting temperature is below about 0° C. Such alcohols are preferably selected from the group consisting of alkyl alcohols having one to five carbons, preferably monoalcohols having one to five carbon atoms, such as methanol, ethanol, n-propanol, isopropanol and combinations thereof.

According to a particularly preferred embodiment, the organic water-miscible organic solvent contains at least 80 wt. %, more preferably at least 90 wt. %, most preferably at least 95 wt. % of C₁₋₅ monoalcohol.

Even more preferably, the water-miscible organic solvent is selected from ethanol, methanol, iso-propanol, n-propanol and combinations thereof. Most preferably, said water-miscible organic solvent is ethanol.

Minor amounts of apolar organic solvents may be present in the present extract, e.g. less than 0.5%, more preferably less than 0.1% by weight of the apolar phase. As explained herein before, it is preferred not to employ such apolar organic solvents. Even more preferably, the extract does not contain any organic solvents besides the water-miscible organic solvent and the oil.

The present extract typically contains a considerable amount of sodium chloride. Typically, the extract contains 0.7-6%, more preferably 0.8-4% of sodium chloride by weight of the polar phase.

The polar phase can typically contain up to 20 wt. % of other water soluble components. As will be understood by those skilled in the art, such other water soluble components, in particular, comprise other water soluble components originating from microalgae. Suitable examples of such components include carbohydrates, proteins, minerals, phospholipids and glycolipids. The polar phase typically contains at least 1 wt. %, more preferably 5 wt. % of such other water soluble components, relative to the weight of the entire polar phase. Usually, the amount of said other water soluble components contained in the polar phase of the extract does not exceed 15 wt. %, preferably it does not exceed 10 wt. %.

The present extracts may optionally comprise other components that are neither contained in the apolar phase nor in the polar phase. A typical example of such other components is cell debris. Said optional other components may be present in an amount that does not exceed 9 wt. % by weight of the water contained in the extract, preferably it does not exceed 8 wt. %, more preferably it does not exceed 7 wt. %.

In accordance with the present invention, the extract does not contain substantial amounts of solid matter, such as cell debris. Preferably, the extract contains less than 7 wt. %, more preferably less than 3 wt. % of cell debris.

Another aspect of the invention relates to a process of extracting oil-soluble pigment from wet biomass of micro-organisms comprising:
combining the wet biomass with a water miscible organic solvent selected from alcohols, acetone and combinations thereof, said biomass containing 10-95 wt. % of water and at least 0.01 wt. %, relative to dry matter weight, of oil-soluble pigment, preferably selected from carotenoids, chlorophylls and combinations thereof;
isolating a liquid polar phase from the combination of biomass and solvent, said polar phase containing 20-80 wt. % of the water miscible organic solvent, 15-75 wt. % of water and at least 0.01 wt. % of pigment originating from the biomass;
adding oil to the liquid polar phase in an amount of 5-150% by weight of the liquid phase to extract oil-soluble pigment from said liquid polar phase into the oil, said oil being selected from triglycerides, diglycerides, monoglycerides, phosphatides, free fatty acids and combinations thereof; and
separating an oil phase containing oil-soluble pigment from the extracted polar phase, said oil phase containing at least 0.05 wt. % of dissolved pigment originating from the biomass.

The term “wet biomass” as used herein encompasses wet biomass that is recovered as such as well as rehydrated dry biomass. The term “wet biomass refers to biomass containing at least 10 wt. % of water. Preferably, the wet biomass contains at least 30 wt. % of water and most preferably more than 50 wt. % of water. Furthermore it is preferred that the water content is less than 95 wt. %, more preferably less than 85 wt. %, most preferably less than 80 wt. %.

The present process offers the important advantage that it employs wet biomass as a starting material which means that drying steps can be avoided. Drying of biomass not only has the disadvantage that it consumes vast quantities of energy, but also has the important drawback that, especially if high temperatures are involved, it may result in deterioration of the end product.

Advantageously, the wet biomass employed in the aforementioned process is a biomass of microalgae, preferably a biomass of a microalgae genus or class selected from Chysophyceae, Xantophyceae, Eustigmatophyceae, Baccilariophyceae, Dinophyceae, Rodophyceae, Phaeophyceae, Chlorophyceae, Prasinophyceae, Cryptophyceae, Botrycoccus, Isochrysis, Tetraselmis, Neochloris, Scenedesmus, Chlorobotrys, Eustigmatos, Pseudostaurastrum, Vischeria, Monodopsis, Ellipsoidion, Pseudocharaciopsis and combinations thereof. Even more preferably, the microalgae employed, belongs to a microalgae genus or class selected from Eustigmatophyceae, Chlorophyceae and combinations thereof.

Since, the present process primarily aims at recovering oil-soluble pigment from wet biomass of micro-organisms, said wet biomass, in a preferred embodiment, contains at least 0.01 wt. %, more preferably 0.1 wt. %, relative to biomass dry matter weight, of oil-soluble pigment, preferably oil-soluble pigment selected from the group of carotenoids, chlorophylls and combinations thereof.

In accordance with one embodiment, the microalgae biomass is subjected to cell disruption before or while being contacted with the extraction solvent in order to maximize recovery of intracellular products. Cell disruption may suitably be achieved by high-pressure homogenization, agitation in the presence of glass and ceramic beads in bead mills, ultrasonication, chemical lysis or by grinding.

The inventors, however, have unexpectedly found that it is not necessary to disrupt the microalgae cells (e.g. by high shear homogenisation) in order to achieve high extraction yields, especially when the water-miscible organic solvent comprises a C₁₋₅ monoalcohol. Therefore, according to another particularly preferred embodiment, the process does not comprise a step of mechanically disrupting the cells contained in the wet biomass prior to the combining of said biomass with the water-miscible organic solvent. An example of mechanical disruption is high shear homogenization, e.g. homogenization at pressures of at least 100 bar. In an even more preferred embodiment of this process the wet microalgae biomass is not subjected to mechanical rupture prior to the isolation of the liquid polar phase.

Also, according to a preferred embodiment, the wet biomass when combined with the water-miscible organic solvent contains at least 10⁸, more preferably at least 10⁹ and most preferably at least 10¹⁰ intact micro-organism cells per gram of wet biomass.

In a preferred embodiment of the present invention an extract is produced by combining the wet biomass with water miscible organic solvent containing at least 70 wt. %, more preferably at least 90 wt. % and most preferably at least 95 wt. % of C₁₋₅ alcohol, especially C₁₋₅ monoalcohol. Preferably, the C₁₋₅ monoalcohol is selected from the group selected from methanol, ethanol, isopropanol, n-propanol and combinations thereof. Most preferably, the C_{1 -5} monoalcohol is ethanol. Most preferably no organic solvents other than C₁₋₅ monoalcohol is added to the wet biomass.

The water-miscible organic solvent is preferably added to the wet biomass in an amount of 10-200 wt. %, relative to the weight of said wet biomass, more preferably in an amount of 50-150 wt. %, most preferably in an amount of 75-125 wt. %. It is furthermore preferred that the water-miscible organic solvents, the biomass and optionally water are combined in such amounts that the weight ratio of water to water-miscible solvent exceeds 1:2, more preferably exceeds 3:4, most preferably exceeds 1:1.

The process of the invention typically comprises a step of blending or mixing the wet biomass and the water-miscible organic solvent, typically by stirring, shaking or the like using standard equipment known to those skilled in the art. Furthermore, the process may comprise a step of holding the blend for a certain period of time, e.g. for at least 2 hours, following the addition of the water-miscible solvent and, optionally, after mixing, so as to allow the composition to settle, e.g. to facilitate separation of the polar phase from the biomass by decantation.

As explained herein before, the present invention provides the important advantage that it does not require application of heat during extraction. There is no merit in employing a temperature higher than 50° C., and desirable results can be obtained by employing an extraction temperature much lower than that. Even though the invention is not necessarily limited in this respect, in a preferred embodiment the temperature during the extraction process does not exceed 50° C., more preferably it does not exceed 45° C., still more preferably it does not exceed 40° C. Most preferably the extraction process is performed at ambient temperatures.

In the above defined process separation of the polar phase comprising water, organic water-miscible solvents and dissolved microalgae components, from the biomass is suitably achieved by centrifugation, filtration, decanting or the like. Most preferably, the polar phase is separated by decanting. The liquid polar phase that is isolated from the combination of biomass and solvent preferably contains not more than 10 wt. %, more preferably not more than 5 wt. % and most preferably not more than 2 wt. % of solid material, such as cell debris.

The isolated liquid polar phase typically contains at least 0.1 wt. % of oil-soluble pigment originating from the biomass. Even more preferably, the liquid polar phase contains 0.5 wt. %, most preferably at least 1 wt. % of said pigment. Usually, the isolated liquid polar phase contains not more than 50 wt. % of the oil-soluble pigment.

In an embodiment of the invention the separation of the liquid polar phase from the biomass may be followed by removal of a portion of the organic water-miscible organic solvent and/or water by means of evaporation. The former can be achieved by employing a variety of techniques, such as evaporation, adsorption, chromatography, solvent extraction etc. In the present process the concentration of water-miscible organic solvent in the isolated liquid phase can be reduced, e.g. by at least 10%, by means of evaporation. Said evaporation may be carried out under reduced pressure and/or elevated temperature. In a preferred embodiment of the invention, the process does not comprise removal of the organic water-miscible solvent.

As a further step, the present process comprises the addition of oil to the liquid polar phase. Said oil preferably contains 50-100 wt. %, more preferably 70-100 wt. %, still more preferably 85-100 wt. %, most preferably 95-100 wt. % of lipids selected from triglycerides, diglycerides, monoglycerides, phosphatides, free fatty acids and combinations thereof. More preferably the oil added to the polar phase comprises at least 50 wt. % of a triglyceride oil selected from vegetable oil, medium chain triglyceride oil and mixtures thereof.

In one particularly preferred embodiment of the invention, the oil that is added to the liquid polar phase comprises at least 50 wt. % of a vegetable oil, more preferably at least 50 wt. % of a vegetable oil selected from the group consisting of corn oil, rapeseed oil, soybean oil, palm oil, sunflower oil and coconut oil, safflower oil, peanut oil, coconut oil, sesame oil, soybean oil, olive oil, cottonseed oil, rice oil, linseed oil or mixtures thereof. It should be understood that these oils and these mixtures of oils may suitably have been hydrogenated and/or interesterified.

The oil is preferably added to the liquid polar phase in an amount of 5-150 wt. %, relative to the weight of said polar phase, more preferably in an amount of 10-125 wt. %, most preferably in an amount of 25-100 wt. %.

The oil phase obtained in the present present process typically contains at least 0.3 wt. %, more preferably at least 1 wt. %, even more preferably at least 2 wt. % and most preferably at least 3 wt. % of dissolved pigment originating from the wet biomass.

In case the biomass employed in the present process comprises microalgae, addition of the oil to the liquid polar phase may suitably yield a microalgae extract as defined herein before.

The process of the invention typically comprises a step of mixing the liquid polar phase and the oil by stirring, shaking or the like using standard equipment known to those skilled in the art. Furthermore, the process may comprise a step of holding the composition obtained by the addition of the oil to the liquid polar phase, e.g. for at least 2 hours, to allow the oil phase and the polar phase to separate.

In a variant of the present process the cell wall components of wet microalgae biomass are enzymatically degraded, optionally followed by removal of non-soluble material, prior to the combining with the water-miscible organic solvent. Enzymatic degradation of the cell walls of the microalgae offers the advantage that it is unnecessary to apply mechanical rupture, notably high shear conditions, to lyse the cells. Thus, in a preferred embodiment of this invention, the process comprises:
combining wet microalgae biomass containing the oil-soluble pigment with an enzyme preparation having cellulase, β-glucanase or β-glucosidase activity; and
allowing the enzyme preparation to degrade the cell walls of the microalgae.

According to a particularly preferred embodiment of the above defined process, the enzyme preparation is combined with the biomass in an amount sufficient to provide at least 1 IU per gram of biomass dry matter of endoglucanase activity and/or at least 0.2 IU per gram of biomass dry matter of units of β-glucanase and/or at least 0.8 IU per gram of biomass dry matter of β-glucosidase activity. Endoglucanase activity is determined using a microplate-based carboxymethylcellulose (CMC) assay as described by Xiao et al (Analytical Biochemistry, 342 (2005), 176-178). One IU of endoglucanase activity is defined as the amount of enzyme that liberates one micromol of reducing sugars (expressed as glucose equivalent) in 1 minute at 50° C. and pH 4.8. One IU of β-glucanase activity is defined as the amount of enzyme that liberates 1 μmol of reducing sugar equivalents (expressed as glucose) per minute at 55° C. and pH 5.0, using β-D-glucan as substrate. Similarly, one IU of β-glucosidase activity is defined as the amount of enzyme that liberates 1 μmol of nitrophenol from para-nitrophenyl-β-D-glucopyranose in 10 minutes at specific assay conditions at 50° C. and pH 4.8.

Best results are obtained in accordance with this embodiment of the invention if a large fraction of the cellulose contained in the original microalgae biomass is hydrolyzed. Advantageously, at least of 50%, more preferably at least 80% of the cellulose is hydrolyzed.

The oil phase containing oil-soluble pigment that is obtained by the present process may be used as such, e.g. in the production of a foodstuffs or animal feed, or it can be downstream processed, e.g. to concentrate the pigment contained therein and/or to remove undesirable components.

In the present process separation of the oil phase from the extracted liquid polar phase is suitably achieved by centrifugation and/or decanting. Most preferably, the oil phase is separated by decanting.

In a preferred embodiment of the invention the isolated and optionally further processed oil phase is encapsulated in a food compatible matrix. Another aspect of the present invention concerns a pigment containing oil extract in the form of an oil phase that can be obtained and preferably is obtained by a process as defined herein before.

The oil extract according to the present invention preferably comprises:
a. 70-99.5 wt. % of oil selected from triglycerides, diglycerides, monoglycerides, phosphatides, free fatty acids and combinations thereof;
b. 0.05-25 wt. % of oil-soluble pigment;
c. up to 30 wt. % of other oil soluble components.

Typically, the oil extract comprises 72-99.5 wt. % of oil, more preferably 75-99 wt. %, even more preferably 80-98 wt. %, and most preferably 85-95 wt. % of oil.

According to another preferred embodiment, the oil contained in the oil extract comprises at least 50 wt. %, more preferably at least 75 wt. % and most preferably at least 90 wt. % of triglycerides.

In one particularly preferred embodiment of the invention, the oil contained in the oil extract comprises at least 50 wt. % of a vegetable oil or medium chain triglyceride oil, more preferably at least 50 wt.% of a vegetable oil selected from the group consisting of corn oil, rapeseed oil, soybean oil, palm oil, sunflower oil and coconut oil, safflower oil, peanut oil, coconut oil, sesame oil, soybean oil, olive oil, cottonseed oil, rice oil, linseed oil or mixtures thereof.

The oil contained in the oil extract typically comprises 1-25 wt. % of an oil of microalgae origin. Marine oil preferably represents less than 50 wt. %, more preferably less than 35 wt. %, and most preferably less than 25 wt. % of the oil contained in the oil extract.

The oil-soluble pigment contained in the oil extract preferably is a pigment selected from the group consisting of carotenoids, chlorophyll and combinations thereof. Besides the latter oil-soluble pigments, other types of oil-soluble pigments may be contained in the oil extract. More preferably, the oil-soluble pigment contained in the present extract is a carotenoid.

The oil extract preferably contains at least 0.3 wt. % of oil-soluble pigments, more preferably at least 0.5 wt. %, still more preferably at least 1.0 wt. %, most preferably at least 3.0 wt. %. Typically, the amount of oil-soluble pigments contained in the extract does not exceed 25 wt. %, more preferably it does not exceed 20 wt. %.

The oil extract preferably contains at least 0.05 wt. % of carotenoids, more preferably at least 0.1 wt. %, still more preferably at least 0.3 wt. %, still more preferably at least 1.0 wt. %, most preferably at least 2.0 wt. %. Typically, the amount of carotenoids contained in the extract does not exceed 25 wt. %, more preferably it does not exceed 20 wt. %.

The oil extract preferably contains not more than 20 wt. % of other oil soluble components, i.e. components other than oil or oil-soluble pigment. The oil extract typically contains at least 0.01 wt. %, more preferably 0.1 wt. % of such other oil soluble components. Usually, said other oil soluble components are contained in the oil extract in a concentration that does not exceed 10 wt. %, more preferably does not exceed 5 wt. %.

Yet another aspect of the invention concerns the use of the microalgae extract of the present invention or the use of an oil phase obtained by the present process in the production of foodstuffs, beverages, nutritional preparations, pharmaceutical preparations, animal feed or cosmetic products. The microalgae extract and the isolated oil phase are particularly suited for use in animal feed as the extract or isolated oil phase can be incorporated into the animal feed without the need of any pretreatment. Consequently, in a particularly preferred embodiment, the extract or the oil phase obtained by the present process is used in the production of animal feed.

Typically, the present use comprises incorporating the extract or the oil phase in an amount that is equivalent to at least 0.1% by weight of the final product.

The invention is further illustrated by means of the following non-limiting examples.

### EXAMPLES

### Example 1

### Materials

Nannochloropsis gaditana Lubian (1982), Division: Heterokontophyta, Class: Eustigmatophyceae. CCAP 849/5 from the Culture Collection of Algae and Protozoa (CCAP)
Isolator: Lubian (pre 1977); Origin: Marine; Cadiz Bay, Cadiz, Spain
Culture: Medium SNA; A; sub
Ethanol 99,9%
Sunflower oil

### Extraction

1) Nannochloropsis microalgae were harvested at their exponential growing phase and the cell suspension was centrifuged to eliminate excess of water with a centrifuge of the type Alfa Laval CLARA 80. Further, the cell slurry was subjected to an additional spray drying process. The moisture content of the resulting cell powder was in the range of 2-6% moisture on dry weight.
2) The algae cell powder was rehydrated by adding amount of water at a ratio 1:1 w:w.
3) The biomass was combined with 100% pure ethanol (2:1 w:w ethanol/wet cell mass).
4) The mixture was stirred for 1 hour at room temperature in erlenmeyer flasks with glass stoppers. The extracts were filtered by suction and the filtrates are collected.
5) Sunflower oil was added (20% of total volume) and the emulsion so obtained was vigorously stirred at room temperature for 2-4 hours.
6) Phase separation was allowed to occur and the coloured oily upper phase was recovered by decantation.
7) The lower (hydroalcoholic phase) was evaporated in a rotational evaporator (65° C., 200 mbar) and the semi-solid residue obtained is weighted.

### Qualitative and Quantitative Analysis

The yield of intermediate step 4 of the aforementioned process was determined by comparing the weight of the dry residue after total evaporation of the solvent to the initial amount of algae biomass.

The total yield of the aforementioned process was determined by comparing the weight of isolated oil phase (having subtracted the amount of added oil) to the amount of dry matter contained in the algae biomass.

The concentration of pigments in the oil samples was measured by thin layer chromatography (TLC) with eluent hexane:acetone 7:3 (v:v), and by subsequently scraping the pigment band corresponding to a known amount of oil extract and measuring differential weight of the silica mass before and after extensive ethanol washing-out of the pigment.

### Results

The TLC pattern of Nannochloropsis extraction showed that pigments are selectively extracted into the oil phase and that other lipids (probably including phospholipids) were largely retained in the water/ethanolic phase.

The extraction of Nannochloropsis with EtOH resulted in 20% extracted product based on the algae initial dry weight.

After the second step of separation of the pigment with sunflower oil; 15% of the initial biomass remained in the water/ethanolic phase. This indicates that roughly 5% of the initial Nannochloropsis biomass was extracted with the sunflower oil phase.

The oil phase extract contained 45.5 mg pigments/g oil as determined by the quantitative TLC determination. Taking into account the original algae biomass extracted, this amount represented a yield of 4% calculated on the initial dry biomass.

### Example 2

### Materials

Haematococcus pluvialis. Flotow (1844), Division: Chlorophyta, Class: Clorophyceae, Order: Volvocales. CCAP 34/6 from the Culture Collection of Algae and Protozoa (CCAP)
Isolator: Droop (1951); Origin: Freshwater; rain pool; Ostpicken Island, Tvarmimme, Finland, Culture: Medium EG:JM; A; cryo. Source Ingrepro BV, NL, Average lipid content: 6-20% depending on harvesting season.
Ethanol 99,9%
Sunflower oil

### Extraction

Haematococcus microalgae were harvested at their exponential growing phase and the cells suspension so obtained was extracted using the same procedures as is described in Example 1.

### Analysis

The pigment containing oil samples so obtained were analyzed in the same way as described in Example 1.

Results and conclusions
The extraction of Haematococcus with ethanol yielded 31% extracted product based on the algae initial dry weight.
The oil phase extract contained 53,5 mg pigments/g oil. Taking into account the original algae biomass extracted, this amount represented a yield of 5% calculated on the initial dry mass.

### Example 3

Example 1 is repeated except that this time the wet biomass of Nannochloropsis microalgae is directly combined with 100% pure ethanol (2:1 w:w ethanol/wet cell mass). The results obtained are comparable to those described in Example 1.

### Example 4

Example 2 is repeated except that this time the wet biomass of Haematococcus microalgae is directly combined with 100% pure ethanol (2:1 w:w ethanol/wet cell mass). The results obtained are comparable to those described in Example 2.

### Comparative Example A

Example 1 was repeated, except that in step 3) the biomass was combined with sunflower oil. The mixture was stirred for 6 hours at different temperatures in erlenmeyer flasks with glass stoppers. The extracts so obtained were filtered and the filtered oil was collected.

It was found that in order to achieve appreciable extraction of oil-soluble pigment, the oil had to be heated to temperatures in excess of 75° C. However, at such elevated temperatures the oil soluble pigments are quickly oxidized. In addition, it turned out that separation of the oil from the biomass was cumbersome.

1-19. (canceled)

20. A microalgae extract consisting of:
(a) 40-95 wt. % of a polar phase comprising:
(i) 15-75 wt. % water;
(ii) 20-80 wt. % of an organic water miscible solvent selected from alcohols, acetone and combinations thereof;
(iii) 0-10 wt. % of sodium chloride;
(iv) up to 40 wt. % of other water soluble components;
(b) 5-60 wt. % of an apolar phase comprising:
(i) 70-99.5 wt. % of oil selected from triglycerides, diglycerides, monoglycerides, phosphatides, free fatty acids and combinations thereof;
(ii) 0.05-25 wt. % of oil-soluble pigment;
(iii) up to 30 wt. % of other oil soluble components; and, optionally
(c) other components in a concentration that does not exceed 9% by weight of the water contained in the extract,
wherein the extract contains less than 0.5% by weight of the apolar phase of apolar organic solvent selected from C₅-C₁₀ alkanes and wherein marine oil represents less than 50 wt. % of the oil contained in the apolar phase.

21. The extract according to claim 20, wherein marine oil represents less than 35 wt. % of the oil contained in the apolar phase.

22. The extract according to claim 20, wherein the oil-soluble pigment is selected from carotenoids, chlorophylls and combinations thereof.

23. The extract according to claim 20, wherein the organic water miscible solvent is selected from the group consisting of alcohols, acetone and mixtures thereof

24. The extract according to claim 23, wherein the organic water miscible solvent is selected from the group consisting of ethanol, methanol, n-propanol, iso-propanol and combinations thereof

25. The extract according to claim 20, wherein the extract contains less than 1 wt. %.

26. The extract according to claim 25, wherein the extract contains less than 0.5 wt. % of the other components.

27. The extract according to claim 20, wherein the water content of the extract does not exceed 30 wt. %.

28. The extract according to claim 20, wherein the oil contained in the apolar phase comprises at least 50 wt.% of a triglyceride oil selected from vegetable oil, medium chain triglyceride oil and mixtures thereof

29. The extract according to claim 20, wherein the polar phase comprises 20-70 wt. % of water and 25-75 wt. % of the water miscible organic solvent.

30. The extract according to claim 20, wherein the organic water miscible solvent and water are contained in the extract in a weight ration that does not exceed 2:1.

31. The extract according to claim 20, wherein the extract comprises 60-90 wt. % of the polar phase and 10-40 wt. % of the apolar phase.

32. A process of extracting oil-soluble pigment from wet biomass of micro-organisms comprising:
(a) combining the wet biomass with a water miscible organic solvent selected from alcohols, acetone and combinations thereof, the biomass comprising 10-95 wt. % of water and at least 0.01% by weight of dry matter of pigment selected from carotenoids, chlorophyls and combinations thereof;
(b) isolating a liquid polar phase from the combination of biomass and solvent, the polar phase comprising 20-80 wt. % of the water miscible organic solvent, 15-75 wt. % of water and at least 0.01 wt. % of pigment originating from the biomass;
(c) adding oil to the liquid polar phase in an amount of 5-150% by weight of the liquid polar phase to extract oil-soluble pigment from the liquid polar phase into the oil, the oil being selected from triglycerides, diglycerides, monoglycerides, phosphatides, free fatty acids and combinations thereof; and
(d) separating an oil phase containing oil-soluble pigment from the extracted polar phase, the oil phase comprising at least 0.05 wt. % of dissolved pigment originating from the biomass.

33. The process according to claim 32, wherein the oil-soluble pigment is selected from carotenoids, chlorophylls and combinations thereof.

34. The process according to claim 32, wherein the organic water miscible solvent is selected from the group consisting of alcohols, acetone and mixtures thereof

35. A pigment-containing oil extract obtainable as the separated oil phase from a process according to claim 32.

36. The oil extract according to claim 35, wherein the extract comprises:
(a) 70-99.5 wt. % of oil selected from triglycerides, diglycerides, monoglycerides, phosphatides, free fatty acids and combinations thereof;
(b) 0.05-25 wt. % of oil-soluble pigment;
(c) up to 30 wt. % of other oil soluble components.

37. The oil extract according to claim 35, wherein marine oil represents less than 50 wt. % of the oil extract.

38. The oil extract according to claim 35, wherein at least 50 wt. % of the oil extract consists of triglyceride oil selected from vegetable oil, medium chain triglyceride oil and mixtures thereof.
